(19) United States
(12) Patent Application Publication
(10) Pub. No.: US 2003/0161871 A1
(43) Pub. Date: Aug. 28, 2003

(54) Solubilized riboflavin

(76) Inventors: Geoffrey Hird, Durham, NC (US); Bill Lambert, Raleigh, NC (US)

Correspondence Address: Choate, Hall & Stewart, Exchange Place, 53 State Street, Boston, MA, 02109, US

(21) Appl. No.: 10/024,876

(22) Filed: Dec. 19, 2001

(51) Int. Cl. A61K 31/724; A61K 31/525; A61K 9/127

(52) U.S. Cl. 424/450; 514/251; 514/58

(57) ABSTRACT

In recognition of the need to facilitate the use of riboflavin as a pharmaceutical and additionally to increase the efficacy of water soluble forms of riboflavin (that may contain precipitated riboflavin), the present invention provides solubilized riboflavin, methods for solubilizing riboflavin and kits comprising solubilized riboflavin.

### BACKGROUND OF THE INVENTION

Sepsis, a major cause of morbidity and mortality in humans and other animals, results from an out-of-control host response to invading microbes. Specifically, sepsis can be triggered by the invasion of these organisms (e.g., bacteria) in the blood, by the toxins produced by these invading organisms, or a combination thereof. Sepsis is most commonly caused by invasion by bacteria, but can also be caused by the invasion of fungi or viruses or virus particles or parasites. This out-of-control host response results from a dramatic rise in the levels cytokines (often in response to the toxins produced by the organisms) and results in an escalation of the clotting cascade throughout the body. Clearly, the systemic invasion of these microorganisms incurs direct damage to tissues, organs and vascular function, and additionally, the toxic components of the microorganisms can lead to rapid systemic inflammatory responses that can quickly damage vital organs and lead to circulatory collapse (septic shock) and oftentimes, death. Specifically, gram negative sepsis is the most common and has a case fatality rate of about 35%. The majority of these infections are caused by Escherichia coli, Klebsiella pneumoniae and Pseudomonas aeruginosa. Gram-positive pathogens such as the staphylococci and streptococci are the second major cause of sepsis. The third major group includes the fungi, with fungal infections causing a relatively small percentage of sepsis cases, but with a high mortality rate.

It has previously been established that, for infections caused by gram-negative bacteria, sepsis is related to the toxic components of the bacteria. Specifically, among the well-described bacterial toxins are the endotoxins or lipopolysaccharides (LPS), a cell-wall structure of the gram-negative bacteria. These molecules are glycolipids that are ubiquitous in the outer membrane of all gram-negative bacteria. While the chemical structure of most of the LPS molecule is complex and diverse, a common feature is the lipid A region of LPS (Rietschel, et al., in the Handbook of Endotoxins, 1:187-214 eds. R. A. Proctor and E. Th. Rietschel, Elsevier, Amsterdam (1984)); recognition of lipid A in biologic systems initiates many, if not all, of the pathophysiologic changes of sepsis. Because lipid A structure is highly conserved among all types of gram-negative organisms, common pathophysiologic changes characterize gram-negative sepsis. It is also generally thought that the distinct cell wall substances of gram-positive bacteria and fungi trigger a similar cascade of events, although the structures involved are not as well studied as gram-negative endotoxin.

Regardless of the etiologic agent, many patients with septicemia or suspected septicemia exhibit a rapid decline over a 24-48 hour period. Thus, rapid methods of diagnosis and treatment delivery are essential for effective patient care. Unfortunately, a confirmed diagnosis as to the type of infection traditionally requires microbiological analysis involving inoculation of blood cultures, incubation for 18-24 hours, plating the causative organism on solid media, another incubation period, and final identification 1-2 days later. Therefore, therapy must be initiated without any knowledge of the type and species of the pathogen, and with no means of knowing the extent of the infection.

Currently, there is no reliable and effective treatment for sepsis and septic shock; rather the only treatment involves the early administration of antibiotics and monitoring of vital signs (e.g., systemic pressure, arterial and venous blood pH, arterial blood gas levels, blood lactate level, renal function, electrolyte levels, etc.) to assess whether progress in combating the infection is being made, or to assess whether life support systems may be necessary. Unfortunately, even with the use of antibiotics, mortality rates for sepsis have not moved from 30-50% range for decades, and incidence is steadily increasing. Recent efforts to develop novel treatments for sepsis have provided some interesting leads; however, most of the attempts at developing a treatment for sepsis have failed mainly due to lack of efficacy (no improvement in survival) (see, Garber, Nature Biotechnol. 2000, 18, 917).

Significantly, it has been discovered that riboflavin and derivatives thereof are useful as immunopotentiating and infection preventing agents and thus are useful in the treatment of sepsis (see, U.S. Pat. Nos. 5,814,632 and 5,945,420). More recently, it has been discovered that high doses of riboflavin and derivatives thereof are particularly useful for the treatment of sepsis. Although certain derivatives of riboflavin (in particular, FMN) are very water soluble, riboflavin itself is insoluble, and thus is more troublesome to administer to a patient. Additionally, even if FMN (5′-monophosphate ester of riboflavin), a water soluble derivative, is utilized, hydrolysis of this compound occurs and results in the formation of ribloflavin as insoluble particles. Clearly, it would be desirable to develop methods for the solubilization and stabilization of riboflavin and derivatives thereof to facilitate its use in the treatment of sepsis and additionally to facilitate other pharmaceutical uses.

### DESCRIPTION OF CERTAIN EMBODIMENTS OF THE INVENTION

In recognition of the need to facilitate the use of riboflavin as a pharmaceutical and additionally to increase the efficacy of water soluble forms of riboflavin (that may contain precipitated riboflavin), the present invention provides solubilized riboflavin, methods for solubilizing riboflavin and kits comprising solubilized riboflavin. It has unexpectedly been discovered that riboflavin, which only has an equilibrium solubility of approximately 70 mcg/mL can be solubilized and thus can be utilized in pharmaceutical preparations. In one aspect of the invention, ribloflavin is solubilized by high concentrations of FMN. In another aspect of the invention, riboflavin is solubilized by a solubilizing agent, which agent includes compositions capable of solubilizing riboflavin or methods for the solubilization of riboflavin.

Riboflavin, which has the structure depicted in Formula I below, is a vitamin that serves a vital role in the metabolism of coenzymes for a wide variety of respiratory flavoproteins.

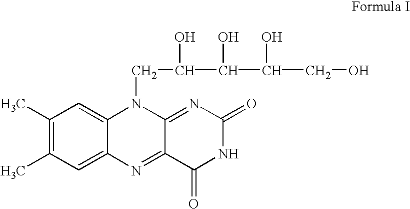

As discussed above, riboflavin only has an equilibrium solubility of approximately 70 mcg/ml and thus presents certain problems for its administration as a pharmaceutical.

In general, the present invention provides a pharmaceutical composition comprising a solubilized form of riboflavin. In certain embodiments, the solubility of riboflavin is greater than the equilibrium solubility of approximately 70 mcg/mL. In certain other embodiments, the solubility of riboflavin is in the range of 100 to about 2000 mcg/mL. In still other embodiments, the solubility of riboflavin is in the range of about 200 to about 1500 mcg/mL.

In one aspect of the invention, solubilization is achieved by the use of high concentrations of FMN. For example, and as shown in the Exemplification herein, high concentrations of FMN result unexpectedly in an increase in the solubility of riboflavin. Thus, in certain embodiments, solutions in the range of 1% to about 10% FMN can be utilized with result in an increase in the intrinsic solubility of riboflavin. In certain embodiments, the concentration of FMN is 5 mg/mL. In certain other embodiments, the concentration is 10 mg/mL and in still other embodiments the concentration is 50 mg/mL. Additionally, it will be appreciated that this effect can be enhanced by changes in pH. For example, pHs in the range of 1 to about 10 can be utilized in the method of the invention. In certain embodiments, pHs in the range of 3 to about 9 are utilized. In certain other embodiments, pHs in the range of about 5 to about 8 are utilized. In yet other embodiments, pHs in the range of 7-8 are utilized.

In yet another aspect of the invention, riboflavin can additionally, or alternatively be solubilized by a solubilizing agent. The term “solubilizing agent”, as used herein, refers to specific compositions (e.g., a complexing agent, liposome, surfactant, co-solvent, oil, emulsion, or microemulsion) capable of solubilizing riboflavin or derivatives thereof, or refers to methods (e.g., solft-gel technology or particle size reduction) utilized to solubilize riboflavin or derivatives thereof. In certain embodiments of the invention, the pharmaceutical composition optionally further comprises a solubilizing agent including, but not limited to: PEG derivatized fatty acids (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxamers, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene) trioleate, sorbitan trioleate, and wheat germ oil, or any combinations thereof.

In certain other embodiments, the composition has is subjected to a procedure to assist solubilization such as particle size reduction or soft gel technology, as described generally in Chapters 13 and 17 of Water-Insoluble Drug Formation, Liu, Ed. Interpharm Press, Denver, Colo., (2000), the entire contents of which are hereby incorporated by reference.

In still another aspect, the present invention also provides a pharmaceutical kit comprising a drug delivery vehicle having at least two compartments, said first compartment comprising riboflavin and optionally a solubilizing agent as described herein and said second compartment comprising a diluent, and/or optionally a solubilizing agent. In certain embodiments, the first compartment comprises riboflavin and said second compartment comprises a solubilizing agent. In certain other embodiments, the first compartment comprises riboflavin and a solubilizing agent and the second compartment comprises a diluent. In still other embodiments, the first compartment comprises in the range of 50 to 2500 mg of riboflavin. In yet other embodiments, the first compartment comprises in the range of 50 to 1000 mg of riboflavin. In certain other embodiments, the first compartment comprises in the range of 50 to 500 mg of riboflavin. In certain embodiments, the kit includes an additional approved therapeutic agent for use as a combination therapy. Optionally associated with such kit can be a notice in the form prescribed by a governmental agency regulating the manufacture, use or sale of pharmaceutical products, which notice reflects approval by the agency of manufacture, use or sale for human administration. In certain embodiments, this kit is in the form of an intravenous bag, a vial or a syringe, examples of which are known in the art. For example, the administration of riboflavin and derivatives thereof, as described above, can be effected by use of the ADD-Vantage® system, as described on page 1396 of the Physicians' Desk Reference, 55^{th} Ed. 2001, Medical Econ. Company, Montvale, N.J., the contents of which are hereby incorporated by reference.

Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance that can be adapted to the practice of this invention in its various embodiments and equivalents thereof.

Exemplification

### EXAMPLE 1

### Formulation

One exemplary embodiment of a formulation and the preparation thereof is shown below. It will be appreciated that, as discussed herein, a broad range of concentration of the active ingredient (riboflavin or derivatives thereof, FMN as shown here) can be utilized. Additionally, the concentration of other ingredients, such as sucrose, can be varied, for example, in the range of 0-20% or more. Additionally, a variety of agents can be substituted in place of sucrose in the example as shown below, and as described more generally herein. For example, a variety of agents could be utilized in place of sucrose including, but not limited to trehalose, lactose, dextrose, PEG, mannitol, and other polyols, and glycine.

| | Amount |
|---|---|
| Component | (mg/vial) |
| E5000 (Riboflavin 5′-Phosphate Sodium) | 419.2 |
| Sucrose | 800.0 |
| Sodium Hydroxide | ca. 23.64 |
| Hydrochloric Acid | qs |
| WFI | ca. 7229 |
| Nitrogen | N/A |
| Total | 8472 |
| Vial, tubing 15 ml, 20 mm, Wheaton, non- | N/A |
| permaglas treated | |
| Stopper, 20 mm 3 leg lyo., Helvoet, | N/A |
| pre-washed, V-9032 | |
| Seal, Flipoff/Tearoff 20 mm, West, White | N/A |

Note: Label content is 400 mg/vial as Riboflavin 5′-monophosphate anhydrous (ratio of molecular weights for monosodium salt to Riboflavin 5′-monophosphate is 1.048). Indicated amount assume a drug substance potency of 100%. HCl and NaOH used for pH adjustment (Target is pH 7.5), quantity will vary with lot. WFI (water for injection) removed during lyophilization, quantity will vary with lot. Nitrogen used in vial headspace (air may also be used).

Depicted more generally below is an exemplary formulation procedure in which sucrose (1.8021 g as shown) is dissolved in water (14 ml as shown). Subsequently FMN (0.9603 g according to the formulation) is added and dissolved. A pH adjustment is performed, qs to 18 ml, and final pH adjustment is performed. The solution is then filtered with a Millex-GV 0.22 micron filter. An initial assay shows 104.3% of 50 mg/ml intent for FMN, and 0.1644% riboflavin (82 mcg/ml) for the formulation described herein.

[image]

### EXAMPLE 2

### Administration

In general, riboflavin and pharmaceutically acceptable derivatives thereof (after an appropriate dosage is determined and formulated) should be injected or infused as soon as possible when the infection can be diagnosed using clinical predictors such as the APACHE score (Knaus, et al., 1991 Chest 100:1619-36 and Knaus et al., 1993 JAMA: 1233-41) or other clinical predictors. In addition, injection or infusion should commence as soon as possible after exposure to endotoxin or diagnosis of systemic gram negative bacterial infection, especially if a more rapid or early diagnostic indicator of systematic gram negative infection becomes available.

In addition, riboflavin and pharmaceutically acceptable derivatives thereof may be administered when exposure to endotoxin can be anticipated. This can occur when:

1) there is an increased probability of elevation of systemic (blood-borne) endotoxin from systemic or localized gram negative bacterial infection (such as during surgery);

2) there is an increased probability that blood levels of endotoxin may increase. In the normal physiological state, endotoxin only minimally translocates across the gut endothelium into splanchnic circulation. This translocated endotoxin is usually then cleared by the liver (and possibly other cells and organs). Increases in blood endotoxin levels can occur when the rate of endotoxin clearance by the liver (or other endotoxin sequestering cells or organs) decreases. Augmentation of gut translocation can occur after gut ischemia, hypoxia, trauma, or other injury to the integrity of the gut lining (or by drug or alcohol toxicity). Blood levels of endotoxin increase when liver function is compromised by disease (cirrhosis), damage (surgery or trauma), or temporary removal (as during liver transplantation);

3) there is an acute or chronic exposure to externally-derived endotoxin resulting in inflammatory response; this exposure can be caused by inhalation or other means of uptake of endotoxin. One example of SIRS (systemic inflammatory response syndrome)-inducing endotoxin uptake is corn dust fever (Schwartz et al., 1994 Am. J. Physiol. 267: L609-17), which affects workers in the grain industry, for example, in the American mid-west. Such workers can be prophylactically treated, e.g., daily, by inhaling an aerosolized formulation of the drug prior to beginning work in, e.g., fields or grain elevators.

For most other prophylactic and therapeutic applications, IV infusion or bolus injection will be used. Injection is most preferable, but infusion may be warranted in some cases by pharmacokinetic requirements.

The treatment should be initiated as soon as possible after diagnosis, and should continue for at least three days, or when assessment of risk of mortality is reduced to an acceptable level.

### EXAMPLE 3

### Solubilization of Riboflavin

The equilibrium solubility of riboflavin is approximately 70 mcg/ml (see plot below with no FMN present). Precipitation is expected to occur if riboflavin reaches a level above this (due to riboflavin in drug substance or due to degradation to riboflavin with time), and precipitation would be unacceptable for patient safety with intravenous use.

Solubility of Riboflavin as a function of pH and % FMN

[image]

Results indicate riboflavin equilibrium solubility increases as a function of FMN concentration (FIG. 1). These data indicate that for 5% FMN, up to 1322 mcg/ml of riboflavin can dissolve at pH 7-8. At 1% FMN, up to 365 mcg/ml of riboflavin can dissolve.

### EXAMPLE 4

### Solubilization of Lumichrome

Like riboflavin, the current formulation also allows lumichrome to dissolve at levels above the equilibrium solubility.

[image]

1. A pharmaceutical composition comprising a solubilized form of riboflavin.

2. The pharmaceutical composition of claim 1, wherein the equilibrium solubility of riboflavin is greater than about 70 mcg/mL.

3. The composition of claim 1, wherein the composition further comprises a solubilizing agent.

4. The composition of claim 1, wherein the solubilizing agent is a complexing agent, liposome, surfactant, co-solvent, oil, emulsion or microemulsion, soft gel technology or particle size reduction.

5. The composition of claim 1, wherein the solubilizing agent is PEG derivatized fatty acid (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxamers, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene) trioleate, sorbitan trioleate, and wheat germ oil, or any combination thereof.

6. A pharmaceutical composition comprising:
riboflavin; and
a solubilizing agent,
wherein the solubilizing agent is a complexing agent, liposome, surfactant, co-solvent, oil, emulsion or microemulsion, soft gel technology or particle size reduction.

7. The pharmaceutical composition of claim 6, wherein the equilibrium solubility of riboflavin is greater than about 70 mcg/mL.

8. The pharmaceutical composition of claim 6, wherein the solubilizing agent is PEG derivatized fatty acid (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxamers, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene) trioleate, sorbitan trioleate, and wheat germ oil, or any combination thereof.

9. A drug delivery vehicle, wherein said vehicle comprises at least two compartments, said first compartment comprising riboflavin and said second compartment comprising a solubilizing agent; and
a means for combining and delivering the contents of the first and second compartments.

10. The drug delivery vehicle of claim 9, wherein said first compartment comprises at least 10 mg of riboflavin and said second compartment comprises a solubilizing agent.

11. The drug delivery vehicle of claim 9, wherein said first compartment comprises at least 50 mg of riboflavin and said second compartment comprises a solubilizing agent.

12. The drug delivery vehicle of claim 9, wherein said first compartment comprises in the range of 50 to 2500 mg of riboflavin and said second compartment comprises a solubilizing agent.

13. The drug delivery vehicle of claim 9, wherein said first compartment comprises in the range of 50 to 1000 mg of riboflavin and said second compartment comprises a solubilizing agent.

14. The drug delivery vehicle of claim 9, wherein said first compartment comprises in the range of 50 to 500 mg of riboflavin and said second compartment comprises a solubilizing agent.

15. The drug delivery vehicle of claim 9, wherein the solubilizing agent is a complexing agent, liposome, surfactant, co-solvent, oil, emulsion or microemulsion, soft gel technology or particle size reduction.

16. The drug delivery vehicle of claim 9, wherein the solubilizing agent is PEG derivatized fatty acid (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxarners, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene) trioleate, sorbitan trioleate, and wheat germ oil, or any combination thereof.

17. The drug delivery vehicle of claim 9, wherein the drug delivery vehicle is an intravenous bag.

18. The drug delivery vehicle of claim 9, wherein the drug delivery vehicle is a vial.

19. The drug delivery vehicle of claim 9, wherein the drug delivery vehicle is a syringe.

20. A drug delivery vehicle, wherein said vehicle comprises at least two compartments, said first compartment comprising riboflavin and a solubilizing agent; and said second compartment comprising a diluent; and
a means for combining and delivering the contents of the first and second compartments.

21. The drug delivery vehicle of claim 20, wherein said first compartment comprises at least 10 mg of riboflavin.

22. The drug delivery vehicle of claim 20, wherein said first compartment comprises at least 50 mg of riboflavin.

23. The drug delivery vehicle of claim 20, wherein said first compartment comprises in the range of 50 to 2500 mg of riboflavin.

24. The drug delivery vehicle of claim 20, wherein said first compartment comprises in the range of 50 to 1000 mg of riboflavin.

25. The drug delivery vehicle of claim 20, wherein said first compartment comprises in the range of 50 to 500 mg of riboflavin.

26. The drug delivery vehicle of claim 20, wherein the solubilizing agent is a complexing agent, liposome, surfactant, co-solvent, oil, emulsion or microemulsion, soft gel technology or particle size reduction.

27. The drug delivery vehicle of claim 20, wherein the solubilizing agent is PEG derivatized fatty acid (e.g., Emulphor), PEG derivatized castor oil (e.g., Cremophore), nicotinamide, nicotinic acid, cyclodextrin (alpha, beta, or gamma) optionally derivatized with sulfobutyl ether or hydroxypropyl groups, liposomes including, but not limited to lecithin or phospholipids, polysorbates, emulphor, poloxamers, sodium dodecyle sulfate, bile salts, polyethylene glycol (PEG), propylene glycol, dimethylacetamide (DMAC), dimethylformamide (DMF), ethanol, N-methylpyrrolidinone, glycerol, lactic acid carbamide, ethyl lactate, dimethylsulfoxide (DMSO), 2-pyrrolidone, dioxolanes, fatty acid esters of glycerol (vegetable oils), ethyl oleate, isopropyl myristate, benzyl benzoate, butyl lactate, 1,3-butylene glycol, castor oil, diethyl carbonate, dimethylacetamide, ethyl acetate, ethyl formate, glycerol monoricinoleate, glyceryl triacetate, isoamyl formate, octyl alcohol, polyoxyethylene oleyl ether, n-propyl alcohol, propylene carbonate, propylene glycol dipelargonate, sesame oil, sorbitan monoisostearate, sorbitan POE (polyoxyethylene) trioleate, sorbitan trioleate, and wheat germ oil, or any combination thereof.

28. The drug delivery vehicle of claim 20, wherein the drug delivery vehicle is an intravenous bag.

29. The drug delivery vehicle of claim 20, wherein the drug delivery vehicle is a vial.

30. The drug delivery vehicle of claim 20, wherein the drug delivery vehicle is a syringe.
